(19) United States
(12) Patent Application Publication
(10) Pub. No.: US 2019/0152248 A1
(43) Pub. Date: May 23, 2019

(54) THERMOSENSITIVE RECORDING MEDIUM AND ARTICLE

(71) Applicants: Kazumasa NODA, Shizuoka (JP); Kenji SHIMIZU, Shizuoka (JP); Yasuhiro KADOTA, Shizuoka (JP)

(72) Inventors: Kazumasa NODA, Shizuoka (JP); Kenji SHIMIZU, Shizuoka (JP); Yasuhiro KADOTA, Shizuoka (JP)

(21) Appl. No.: 16/313,325

(22) PCT Filed: Jun. 26, 2017
(86) PCT No.: PCT/JP2017/023453

§ 371 Date: Dec. 26, 2018

(30) Foreign Application Priority Data
Jun. 28, 2016 (JP) 2016-127948
May 11, 2017 (JP) 2017-094327
Jun. 23, 2017 (JP) 2017-123262

(51) Int. Cl. B41M 5/333; B41M 5/155; B41M 5/327; C07C 317/22

(52) U.S. Cl. CPC B41M 5/3336; C07C 317/22; B41M 5/3275; B41M 5/155

(57) ABSTRACT

Provided is a thermosensitive recording medium including a support, and a thermosensitive recording layer provided over at least one surface of the support and containing a leuco dye and a developer, wherein the developer includes 4-hydroxy-4′-benzyloxydiphenylsulfone and a diphenylsulfone compound represented by general formula (1), and wherein a content of the diphenylsulfone compound represented by general formula (1) is less than 3.0 parts by mass relative to 1.0 part by mass of the 4-hydroxy-4′-benzyloxydiphenylsulfone.

### TECHNICAL FIELD

The present invention relates to a thermosensitive recording medium and an article.

### BACKGROUND ART

Thermosensitive recording methods using thermosensitive recording media are advantageous over other recording methods in that there is no need for, for example, developing and fixing processes, recording is available in a short time using apparatuses having relatively simple configurations, and costs taken are low. Therefore, the thermosensitive recording media are widely used in many fields such as the field of POS for, for example, perishable foods, box lunches, and delicatessen, the field of copying of, for example, books and documents, the field of communication by, for example, facsimile, the field of ticketing of, for example, receipts and signed receipts by ticketing machines, and baggage tags in the airline industry.

Compared with the past, there are significantly increasing opportunities in which the surfaces of tickets for public transportation and commuter passes using the thermosensitive recording media are touched by fingers due to popularization of automatic ticket gates. Therefore, there occurs a new problem that hand creams used for hand protection adhere to the thermosensitive recording layers of the tickets and the commuter passes, causing bad influences such as degradation of the hardness or fading of the image portions.

Further, there are cases where thermosensitive recording labels formed of the thermosensitive recording media are used by being pasted on pill bottles and pill cases used in, for example, drug stores. The pill bottles and the pill cases are assumed to be stored in various places in the households, and may be touched by hands to which hand creams used for hand protection have been applied. Here, there is the new problem that the image portions touched by the hand creams may fade.

Hence, there is proposed a thermosensitive recording medium containing a hydrazine self-cross-linking acrylic-based resin as a binder in the thermosensitive recording layer in order to have, for example, an improved hand cream resistance, an improved laundering fastness, and an improved heat resistance (see, e.g., PTL 1).

### CITATION LIST

### Patent Literature

[PTL 1] Japanese Unexamined Patent Application Publication No. 07-117354

### SUMMARY OF INVENTION

### Technical Problem

The present invention has an object to provide a thermosensitive recording medium having a high color developing sensitivity, a high image density, and an excellent hand cream resistance.

### Solution to Problem

According to one aspect of the present invention, a thermosensitive recording medium includes a support and a thermosensitive recording layer provided over at least one surface of the support and containing a leuco dye, a first developer, and a second developer. The first developer and the second developer are 4-hydroxy-4′-benzyloxydiphenylsulfone and a diphenylsulfone compound represented by the following general formula (1), respectively. The content of the diphenylsulfone compound represented by general formula (1) is less than 3.0 parts by mass relative to 1.0 part by mass of the 4-hydroxy-4′-benzyloxydiphenylsulfone.

<General Formula (1)>

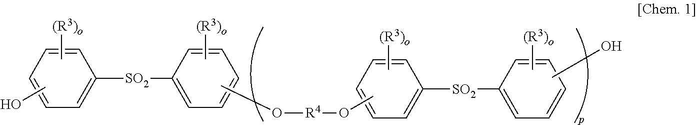

In general formula (1), R³ represents any one of a hydrogen atom, a halogen atom, an alkyl group containing from 1 through 6 carbon atoms, and an alkenyl group containing from 1 through 6 carbon atoms, each o independently represents an integer of from 0 through 4, p represents an integer of from 1 through 11, and R⁴ represents any one of a saturated or unsaturated, straight-chained or branched hydrocarbon group that contains from 1 through 12 carbon atoms and may contain an ether bond, a substituted phenylene group represented by the following general formula (1-1), and a divalent group represented by the following general formula (1-2).

<General Formula (1-1)>

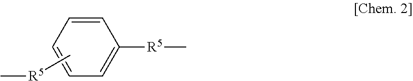

In general formula (1-1), R⁵ represents any one of a methylene group and an ethylene group.

<General Formula (1-2)>

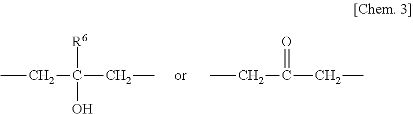

In general formula (1-2), R⁶ represents any one of a hydrogen atom and an alkyl group containing from 1 through 4 carbon atoms.

### Advantageous Effects of Invention

The present invention can provide a thermosensitive recording medium having a high color developing sensitivity, a high image density, and an excellent hand cream resistance.

### DESCRIPTION OF EMBODIMENTS

(Thermosensitive Recording Medium)

A thermosensitive recording medium of the present invention includes a support and a thermosensitive recording layer on at least one surface of the support, preferably includes an undercoat layer and a protective layer, and further includes other layers as needed.

The thermosensitive recording medium of the present invention is based on the following finding. Existing techniques prevent the surfaces of the thermosensitive recording media from degradation of hardness, i.e., hand cream resistance, with addition of a hydrazine self-cross-linking acrylic-based resin as a binder in the thermosensitive recording layers. However, the existing techniques cannot prevent image degradation due to adhesion of hand creams on the image portions.

Hence, the first developer and the second developer contained in the thermosensitive recording layer of the thermosensitive recording medium of the present invention are 4-hydroxy-4′-benzyloxydiphenylsulfone and a diphenylsulfone compound represented by the following general formula (1), respectively. The content of the diphenylsulfone compound represented by general formula (1) is less than 3.0 parts by mass relative to 1.0 part by mass of the 4-hydroxy-4′-benzyloxydiphenylsulfone. In this way, a high color developing sensitivity, a high image density, and an excellent hand cream resistance can be realized.

Examples of the hand cream related with the hand cream resistance include THERAPY DAILY MOISTURE available from Curel Advanced Ceramide and containing glycerin as a moisturizing component.

The specific mechanism of the present invention allowing the excellent hand cream resistance is unknown. However, it is estimated that combined use of specific 2 developers having a low solubility in alcohols enables a reaction product of a leuco dye and the developers to have resistance against hand creams mainly formed of polyvalent alcohols such as glycerin.

<Thermosensitive Recording Layer>

The thermosensitive recording layer contains a leuco dye, a developer, and a binder resin, and further contains other components as needed.

—Leuco Dye—

The leuco dye is not particularly limited and may be appropriately selected depending on the intended purpose from leuco dyes used in thermosensitive recording media. Preferable examples of the leuco dye include leuco compounds such as triphenylmethane-based, fluoran-based, phenothiazine-based, auramine-based, spiropyran-based, and indolinophthalide-based dyes.

The leuco dye is not particularly limited and may be appropriately selected depending on the intended purpose. Examples of the leuco dye include 3,3-bis(p-dimethylaminophenyl)-phthalide, 3,3-bis(p-dimethylaminophenyl)-6-dimethylaminophthalide (also known as crystal violet lactone), 3,3-bis(p-dimethylaminophenyl)-6-diethylaminophthalide, 3,3-bis(p-dimethylaminophenyl)-6-chlorophthalide, 3,3-bis(p-dibutylaminophenyl)phthalide, 3-c yclohexylamino-6-chlorofluoran, 3-dimethylamino-5,7-dimethylfluoran, 3-diethylamino-7-chlorofluoran, 3-diethylamino-7-methylfluoran, 3-diethylamino-7,8-benzfluoran, 3-diethylamino-6-methyl-7-chlorofluoran, 3-(N-p-tolyl-N-ethylamino)-6-methyl-7-anilinofluoran, 2-{N-(3′-trifluoromethylphenyl)amino}-6-diethylaminofluoran, 2-{3,6-bis(diethylamino)-9-(o-chloroanilino)xanthyl lactam benzoate}, 3-diethylamino-6-methyl-7-(m-trichloromethylanilino)fluoran, 3-diethylamino-7-(o-chloroanilino)fluoran, 3-pyrrolidino-6-methyl-7-anilinofluoran, 3-di-n-butylamino-7-o-chloroanilino)fluoran, 3-N-methyl-N,n-amylmino-6-methyl-7-anilinofluoran, 3-N-methyl-N-cyclohexylamino-6-methyl-7-anilinofluoran, 3-diethylamino-6-methyl-7-anilinofluoran, 3-(N,N-diethylamino)-5-methyl-7-(N,N-dibenzylamino)fluoran, benzoyl leuco methylene blue, 6′-chloro-8′-methoxy-benzoindolino-spiropyran, 6′-bromo-3′-methoxy-benzoindolino-spiropyran, 3-(2′-hydroxy-4′-dimethylaminophenyl)-3-(2′-methoxy-5′ chlorophenyl)phthalide, 3-(2′-hydroxy-4′-dimethylaminophenyl)-3-(2′-methoxy-5′-nitrophenyl)phthalide, 3-(2′-hydroxy-4′-diethylaminophenyl)-3-(2′-methoxy-5′-methylphenyl)phthalide, 3-(2′-methoxy-4′-dimethylaminophenyl)-3-(2′-hydroxy-4′-chloro-5′-methylphenyl)phthalide, 3-(N-ethyl-N-tetrahydrofurfuryl)amino-6-methyl-7-anilinofluoran, 3-N-ethyl-N-(2-ethoxypropyl)amino-6-methyl-7-anilinofluoran, 3-N-methyl-N-isobutyl-6-methyl-7-anilinofluoran, 3-morpholino-7-(N-propyl-trifluoromethylanilino)fluoran, 3-pyrrolidino-7-trifluoromethylanilinofluoran, 3-diethylamino-5-chloro-7-(N-benzyl-trifluoromethylanilino)fluoran, 3-pyrrolidino-7-(di-p-chlorophenyl)methylaminofluoran, 3-diethylamino-5-chloro-7-(α-phenylethylamino)fluoran, 3-(N-ethyl-p-toluidino)-7-(α-phenylethylamino)fluoran, 3-diethylamino-7-(o-methoxycarbonylphenylamino)fluoran, 3-diethylamino-5-methyl-7-(α-phenylethylamino)fluoran, 3-diethylamino-7-piperidinofluoran, 2-chloro-3-(N-methyltoluidino)-7-(p-n-butylanilino)fluoran, 3-di-n-butylamino-6-methyl-7-anilinofluoran, 3,6-bis(dimethylamino)fluorenespiro(9,3′)-6′-dimethylaminophthalide, 3-(N-benzyl-N-cyclohexylamino)-5,6-benzo-7-α-naphthylamino-4′-bromofluoran, 3-diethylamino-6-chloro-7-anilinofluoran, 3-diethylamino-6-methyl-7-mesitidino-4′,5′-benzofluoran, 3-N-methyl-N-isopropyl-6-methyl-7-anilinofluoran, 3-N-ethyl-N-isoamyl-6-methyl-7-anilinofluoran, 3-diethylamino-6-methyl-7-(2′,4′-dimethylanilino)fluoran, 3-morpholino-7-(N-propyl-trifluoromethylanilino)fluoran, 3-pyrrolidino-7-trifluoromethylanilinofluoran, 3-diethylamino-5-chloro-7-(N-benzyl-trifluoromethylanilino)fluoran, 3-pyrrolidino-7-(di-p-chlorophenyl)methylaminofluoran, 3-diethylamino-5-chloro-(α-phenylethylamino)fluoran, 3-(N-ethyl-p-toluidino)-7-(α-phenylethylamino)fluoran, 3-diethylamino-7-(o-methoxycarbonylphenylamino)fluoran, 3-diethylamino-5-methyl-7-(α-phenylethylamino)fluoran, 3-diethylamino-7-piperidinofluoran, 2-chloro-3-(N-methyltoluidino)-7-(p-N-butylanilino)fluoran, 3,6-bis(dimethylamino)fluorenespiro(9,3′)-6′-dimethylaminophthalide, 3-(N-benzyl-N-cyclohexylamino)-5,6-benzo-7-α-naphthylamino-4′-bromofluoran, 3-diethylamino-6-chloro-7-anilinofluoran, 3-N-ethyl-N-(-2-ethoxypropyl)amino-6-methyl-7-anilinofluoran, 3-N-ethyl-N-tetrahydrofurfurylamino-6-methyl-7-anilinofluoran, 3-diethylamino-6-methyl-7-mesitidino-4′,5′-benzofluoran, 3-p-dimethylaminophenyl)-3-{1,1 -bis(p-dimethylaminophenyl)ethylen-2-yl}phthalide, 3-(p-dimethylaminophenyl)-3-{1,1-bis (p-dimethylaminophenyl)ethylen-2-yl}-6-dimethylaminophthalide, 3-(p-dimethylaminophenyl)-3-(1-p-dimethylaminophenyl-1-phenylethylen-2-yl)phthalide, 3-(p-dimethylaminophenyl)-3-(1-p-dimethylaminophenyl-1-p-chlorophenylethylen-2-yl)-6-dimethylaminophthalide, 3-(4′-dimethylamino-2′-methoxy)-3-(1″-p-dimethylaminophenyl-1″-p-chlorophenyl-1″, 3″-butadien-4″-yl)benzophthalide, 3-(4′-dimethylamino-2′-benzyloxy)-3-(1″-p-dimethylaminophenyl-1″-phenyl-1″,3″-butadien-4″-yl)benzophthalide, 3-dimethylamino-6-dimethylamino-fluorene-9-spiro-3′-(6′-dimethylamino)phthalide, 3,3-bis (2-(p-dimethylaminophenyl)-2-p-methoxyphenyl)ethenyl)-4,5,6,7-tetrachlorophthalide, 3-bis{1,1-bis(4-pyrrolidinophenyl)ethylen-2-yl}-5,6-dichloro-4,7-dibromophthalide, bis(p-dimethylaminostyryl)-1-naphthalene sulfonylmethane, and bis(p-dimethylaminostyryl)-1-p-trisulfonylmethane. One of these leuco dyes may be used alone or two or more of these leuco dyes may be used in combination.

—Developer—

The first developer and the second developer are 4-hydroxy-4′-benzyloxydiphenylsulfone and a diphenylsulfone compound represented by the following general formula (1), respectively.

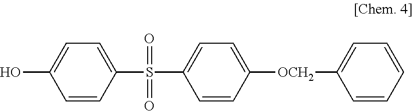

<General Formula (1)>

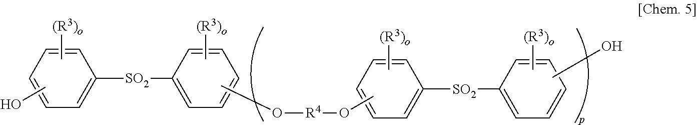

In general formula (1), R³ represents any one of a hydrogen atom, a halogen atom, an alkyl group containing from 1 through 6 carbon atoms, and an alkenyl group containing from 1 through 6 carbon atoms, each o independently represents an integer of from 0 through 4, p represents an integer of from 1 through 11, and R⁴ represents any one of a saturated or unsaturated, straight-chained or branched hydrocarbon group that contains from 1 through 12 carbon atoms and may contain an ether bond, a substituted phenylene group represented by general formula (1-1) below, and a divalent group represented by the following general formula (1-2).

<General Formula (1-1)>

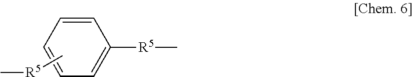

In general formula (1-1), R⁵ represents any one of a methylene group and an ethylene group.

<General Formula (1-2)>

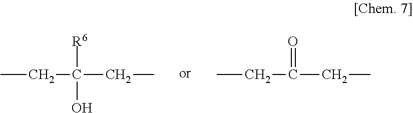

In general formula (1-2), R⁶ represents any one of a hydrogen atom and an alkyl group containing from 1 through 4 carbon atoms.

Each R³ may be the same or different, is preferably the same, represents any one of a hydrogen atom, a halogen atom, an alkyl group containing from 1 through 6 carbon atoms, and an alkenyl group containing from 1 through 6 carbon atoms, and preferably represents a hydrogen atom.

The alkyl group or the alkenyl group is an alkyl group or an alkenyl group containing from 1 through 6 carbon atoms, and examples of such an alkyl group and an alkenyl group include a methyl group, an ethyl group, an n-propyl group, an isopropyl group, an n-butyl group, a sec-butyl group, a tert-butyl group, an n-pentyl group, an isopentyl group, a neopentyl group, a tert-pentyl group, an n-hexyl group, an isohexyl group, a 1-methylpentyl group, a 2-methylpentyl group, a vinyl group, an allyl group, an iso-propenyl group, a 1-propenyl group, a 2-butenyl group, a 3-butenyl group, a 1,3-butanedienyl group, and a 2-methyl-2-propenyl group.

Examples of the halogen atom include a chlorine atom, a bromine atom, a fluorine atom, and an iodine atom. Among these halogen atoms, a chlorine atom and a bromine atom are preferable.
Each o is independently from 0 through 4, and preferably represents 0.
It is preferable that the OH group and the —OR⁴O— group be at the para position of the SO₂ group.
It is preferred to use a mixture of diphenylsulfone compounds, each of which is represented by the general formula (1) and is different in above mentioned value of p.

R⁴ may be the same or different, but is preferably the same.

R⁴ may be a saturated or unsaturated, preferably saturated, straight-chained or branched, preferably straight-chained hydrocarbon group that contains from 1 through 12, preferably from 3 through 7 carbon atoms, and may contain an ether bond. Preferable examples of such a hydrocarbon include a polyalkylene oxide chain and an alkylene group. A polyalkylene oxide chain is preferable.

Examples of the polyalkylene oxide chain (—OR⁴O—) include —O—(CₐH₂ₐO)_{1,3}— (where a is from 2 through 4, preferably from 2 through 3, and more preferably 2). Examples of the alkylene group include —C_{b}H_{2b}— (where b represents an arbitrary integer).

R⁴ may represent a substituted phenylene group represented by the following general formula (1-1) or a divalent group represented by the following general formula (1-2).

<General Formula (1-1)>

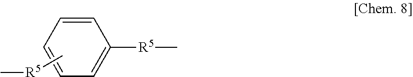

In general Formula (1-1), R⁵ represents any one of a methylene group and an ethylene group.

<General Formula (1-2)>

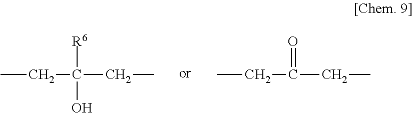

In general Formula (1-2), R⁶ represents any one of a hydrogen atom and an alkyl group containing from 1 through 4 carbon atoms.

Among these examples, R⁴ is preferably the saturated or unsaturated, straight-chained or branched hydrocarbon group that contains from 1 through 12 carbon atoms and may contain an ether bond.

Examples of groups represented by R⁴ in the diphenylsulfone compound include a methylene group, an ethylene group, a trimethylene group, a tetramethylene group, a pentamethylene group, a hexamethylene group, a heptamethylene group, an octamethylene group, a nonamethylene group, a decamethylene group, an undecamethylene group, a dodecamethylene group, a methyl methylene group, a dimethyl methylene group, a methyl ethylene group, a methylene ethylene group, an ethyl ethylene group, a 1,2-dimethyl ethylene group, a 1-methyl trimethylene group, a 1-methyl tetramethylene group, a 1,3-dimethyl trimethylene group, a 1-ethyl-4-methyl-tetramethylene group, a vinylene group, a propenylene group, a 2-butenylene group, an ethynylene group, a 2-butynylene group, a 1-vinyl ethylene group, an ethylene oxyethylene group, a tetramethylene oxytetramethylene group, an ethylene oxyethylene oxyethylene group, an ethylene oxymethylene oxyethylene group, a 1,3-dioxane-5,5-bismethylene group, a 1,2-xylyl group, a 1,3-xylyl group, a 1,4-xylyl group, a 2-hydroxytrimethylene group, a 2-hydroxy-2-methyl trimethylene group, a 2-hydroxy-2-ethyl trimethylene group, a 2-hydroxy-2-propyl trimethylene group, a 2-hydroxy-2-isopropyl trimethylene group, and a 2-hydroxy-2-butyl trimethylene group.

The diphenylsulfone compound that may be used may be a mixture of some kinds of diphenylsulfone compounds varied in at least any one of the substituent (R³) and p. The content ratios of these kinds of diphenylsulfone compounds are optional. Examples of the mixing method include, but are not particularly limited to, mixing in the form of powders, mixing in the form of dispersion liquids in, for example, water, and a method of simultaneously producing a plurality of kinds of diphenylsulfone compounds depending on production conditions such that the plurality of kinds of diphenylsulfone compounds are contained in the mixture.

Examples of the compound represented by general formula (1) include: 4,4′-bis[4-[4-(4-hydroxyphenylsulfonyl)phenoxy]-2-trans-butenyloxy]diphenylsulfone; 4,4′-bis[4-(4-hydroxyphenylsulfonyl)phenoxy-4-butyloxy]diphenylsulfone; 4,4′-bis[4-(4-hydroxyphenylsulfonyl)phenoxy-3-propyloxy]diphenylsulfone; 4,4′-bis [4-(4-hydroxyphenylsulfonyl)phenoxy-2-ethyloxy]diphenylsulfone; 4-[4-(4-hydroxyphenylsulfonyl)phenoxy-4-butyloxy]-4′-[4-(4-hydroxyphenylsulfonyl) phenoxy-3-propyloxy] diphenylsulfone; 4-[4-(4-hydroxyphenylsulfonyl)phenoxy-4-butyloxy]-4′-[4-(4-hydroxyphenylsulfonyl) phenoxy-2-ethyloxy]diphenylsulfone; 4-[4-(4-hydroxyphenylsulfonyl)phenoxy-3-propyloxy]-4′-[4-(4-hydroxyphenylsulfonyl) phenoxy-2-ethyloxy]diphenylsulfone; 4,4′-bis[4-(4-hydroxyphenylsulfonyl)phenoxy-5-pentyloxy]diphenylsulfone; 4,4′-bis[4-(4-hydroxyphenylsulfonyl)phenoxy-6-hexyloxy]diphenylsulfone; 4-[4-[4-(4-hydroxyphenylsulfonyl)phenoxy]-2-trans-butenyloxy]-4′-[4-(4-hydroxyphenylsulfonyl)phenoxy-4-butyloxy]diphenylsulfone; 4-[4-(4-hydroxyphenylsulfonyl)phenoxy-2-trans-butenyloxy]-4′-[4-(4-hydroxyphenylsulfonyl)phenoxy-3-propyloxy]diphenylsulfone; 4-[4-[4-(4-hydroxyphenylsulfonyl)phenoxy]-2-trans-butenyloxy]-4′-[4-(4-hydroxyphenylsulfonyl)phenoxy-2-ethyloxy]diphenylsulfone; 1,4-bis[4-[4-[4-(4-hydroxyphenylsulfonyl)phenoxy-2-trans-butenyloxy]phenylsulfonyl ]phenoxy]-cis-2-butene; 1,4-bis[4-[4-[4-(4-hydroxyphenylsulfonyl)phenoxy-2-trans-butenyloxy]phenylsulfonoyl]phenoxy]-trans-2-butene; 4,4′-bis[4-[4-(2-hydroxyphenylsulfonyl)phenoxy]butyloxy]diphenylsulfone; 4,4′-bis[4-[2-(4-hydroxyphenylsulfonyl)phenoxy]butyloxy]diphenylsulfone; 4,4′-bis[4-(4-hydroxyphenylsulfonyl)phenoxy-2-ethyleneoxyethoxy]diphenylsulfone; 4,4′-bis[4-(4-hydroxyphenylsulfonyl)phenyl-1,4-phenylenebismethyleneoxy]diphenylsulfone; 4,4′-bis[4-(4-hydroxyphenylsulfonyl)phenyl-1,3-phenylenebismethyleneoxy]diphenylsulfone; 4,4′-bis[4-(4-hydroxyphenylsulfonyl)phenyl-1,2-phenylenebismethyleneoxy]diphenylsulfone; 2,2′-bis[4-[4-[4-(4-hydroxyphenylsulfonyl)phenoxy-2-ethyleneoxyethoxy]phenylsulfonyl]phenoxy]diethyl ether; α,α′-bis[4-[4-[4-(4-hydroxyphenylsulfonyl)phenyl-1,4-phenylenebismethyleneoxy]phenylsulfonyl]phenoxy]-p-xylene; α,α′-bis[4-[4-[4-(4-hydroxyphenylsulfonyl)phenyl-1,3-phenylenebismethyleneoxy]phenylsulfonyl]phenoxy]-m-xylene; α,α′-bis[4-[4-[4-(4-hydroxyphenylsulfonyl)phenyl-1,2-phenylenebismethyleneoxy]phenylsulfonyl]phenoxy]-o-xylene; 2,4′-bis[2-(4-hydroxyphenylsulfonyl)phenoxy-2-ethyleneoxyethoxy]diphenylsulfone; 2,4′-bis[4-(2-hydroxyphenylsulfonyl)phenoxy-2-ethyleneoxyethoxy]diphenylsulfone; 4,4′-bis[3,5-dimethyl-4-(3,5-dimethyl-4-hydroxyphenylsulfonyl)phenoxy-2-ethyleneoxyethoxy]diphenylsulfone; 4,4′-bis[3-allyl-4-(3-allyl-4-hydroxyphenylsulfonyl)phenoxy-2-ethyleneoxyethoxy]diphenylsulfone; 4,4′-bis[3,5-dimethyl-4-(3,5-dimethyl-4-hydroxyphenylsulfonyl)phenyl-1,4-phenylene bismethyleneoxy]diphenylsulfone; 4,4′-bis[3,5-dimethyl-4-(3,5-dimethyl-4-hydroxyphenylsulfonyl)phenyl-1,3-phenylene bismethyleneoxy]diphenylsulfone; 4,4′-bis[3,5-dimethyl-4-(3,5-dimethyl-4-hydroxyphenylsulfonyl)phenyl-1,2-phenylene bismethyleneoxy]diphenylsulfone; 4,4′-bis[3-allyl-4-(3-allyl-4-hydroxyphenylsulfonyl)1,4-phenylenebismethyleneoxy]diphenylsulfone; 4,4′-bis[3-allyl-4-(3-allyl-4-hydroxyphenylsulfonyl)1,3-phenylenebismethyleneoxy]diphenylsulfone; 4,4′-bis[3-allyl-4-(3-allyl-4-hydroxyphenylsulfonyl)1,2-phenylenebismethyleneoxy]diphenylsulfone; 4,4′-bis[4-(4-hydroxyphenylsulfonyl)phenoxy-2-hydroxypropyloxy]diphenylsulfone; and 1,3-bis[4-[4-[4-(4-hydroxyphenylsulfonyl)phenoxy-2-hydroxypropyloxy]phenylsulfonyl]phenoxy]-2-hydroxypropane. One of these compounds may be used alone or two or more of these compounds may be used in combination.

When some kinds of diphenylsulfone compounds each represented by general formula (1) are used as a mixture, a particularly preferable composition of the mixture contains 2 or more kinds of diphenylsulfone compounds in which R³ is the same and only p takes different values. Such compounds can be produced by a simple method, and it is possible to simultaneously synthesize compounds having different p values at arbitrary content ratios, by varying the reaction ratios of the materials. Among such compounds, particularly, examples of compounds represented by general formula (1) where p=1 include 1,3-bis[4-(4-hydroxyphenylsulfonyl)phenoxy]-2-hydroxypropane, 1,1-bis[4-(4-hydroxyphenylsulfonyl)phenoxy]methane, 1,2-bis[4-(4-hydroxyphenylsulfonyl)phenoxy]ethane, 1,3-bis[4-(4-hydroxyphenylsulfonyl)phenoxy]propane, 1,4-bis[4-(4-hydroxyphenylsulfonyl)phenoxy]butane, 1,5-bis[4-(4-hydroxyphenylsulfonyl)phenoxy]pentane, 1,6-bis[4-(4-hydroxyphenylsulfonyl)phenoxy]hexane, α,α′-bis[4-(4-hydroxyphenylsulfonyl)phenoxy]-p-xylene, α,α′-bis[4-(4-hydroxyphenylsulfonyl)phenoxy]-m-xylene, α,α′-bis[4-(4-hydroxyphenylsulfonyl)phenoxy]-o-xylene, 2,2′-bis[4-(4-hydroxyphenylsulfonyl)phenoxy]diethyl ether, 4,4′-bis[4-(4-hydroxyphenylsulfonyl)phenoxy]dibutyl ether, 1,2-bis[4-(4-hydroxyphenylsulfonyl)phenoxy]ethylene, and 1,4-bis[4-(4-hydroxyphenylsulfonyl)phenoxy]-2-butene (see, e.g., Japanese Unexamined Patent Application Publication No. 07-149713, International Publication No. WO 93/06074, and International Publication No. WO 95/33714).

As such a compound, a commercially available product named D90 (available from Nippon Soda Co., Ltd.) represented by the following structural formula A can be used.

<Structural Formula A>

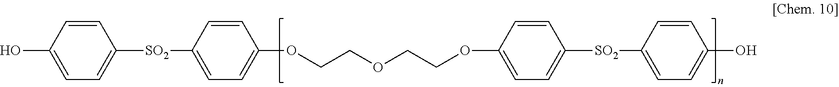

In structural formula A, n is a numerical value of from 1 to 11.

The product is a mixture of compounds represented by structural formula A where n is a numerical value of from 1 to 11.

The content of the diphenylsulfone compound represented by general formula (1) is less than 3.0 parts by mass, preferably 0.1 parts by mass or greater but 2.5 parts by mass or less, more preferably 0.3 parts by mass or greater but 1.5 parts by mass or less, and still more preferably 0.3 parts by mass or greater but 1.0 part by mass or less relative to 1.0 part by mass of the 4-hydroxy-4′-benzyloxydiphenylsulfone. When the content of the diphenylsulfone compound represented by general formula (1) is less than 3.0 parts by mass relative to 1.0 part by mass of the 4-hydroxy-4′-benzyloxydiphenylsulfone, a thermosensitive recording medium having a high color developing sensitivity, a high image density, and an excellent hand cream resistance can be obtained.

In addition to the 4-hydroxy-4′-benzyloxydiphenylsulfone and the diphenylsulfone compound represented by general formula (1), the developer may further include any other known developer so long as the object and effect of the present invention are achieved.

The content of the developer is not particularly limited, may be appropriately selected depending on the intended purpose, and is preferably 1 part by mass or greater but 20 parts by mass or less and more preferably 2 parts by mass or greater but 10 parts by mass or less relative to 1 part by mass of the leuco dye.

—Binder Resin—

The binder resin is not particularly limited and may be appropriately selected depending on the intended purpose. Examples of the binder resin include: polyvinyl alcohol resins, and starch or derivatives of starch; cellulose derivatives such as hy-droxymethyl cellulose, hydroxyethyl cellulose, carboxymethyl cellulose, methyl cellulose, and ethyl cellulose; water-soluble polymers such as sodium polyacrylate, polyvinyl pyrrolidone, acrylamide-acrylic acid ester copolymers, acrylamide-acrylic acid ester-methacrylic acid terpolymers, alkali salts of styrene-maleic anhydride copolymers, alkali salts of isobutylene-maleic anhydride copolymers, polyacrylamide, sodium alginate, gelatin, and casein; emulsions of, for example polyvinyl acetate, polyurethane, polyacrylic acid, polyacrylic acid ester, vinyl chloride-vinyl acetate copolymers, polybutyl methacrylate, and ethylene-vinyl acetate copolymers; and latexes of, for example, styrene-butadiene copolymers and styrene-butadiene-acrylic copolymers. One of these binder resins may be used alone or two or more of these binder resins may be used in combination. Among these binder resins, styrene-butadiene copolymers (SBR) are particularly preferable in terms of improving water resistance.

As needed, various thermally fusible substances may be added in the thermosensitive recording layer as sensitivity improvers.

The thermally fusible substances are not particularly limited and may be appropriately selected depending on the intended purpose. Examples of the thermally fusible substances include: fatty acids such as stearic acid and behenic acid; fatty acid amides such as stearic acid amide and palmitic acid amide; fatty acid metal salts such as zinc stearate, aluminum stearate, calcium stearate, zinc palmitate, and zinc behenate; and p-benzyl biphenyl, terphenyl, triphenylmethane, benzyl p-benzyloxybenzoate, β-benzyloxynaphthalene, phenyl β-naphthoate, phenyl 1-hydroxy-2-naphthoate, methyl 1-hydroxy-2-naphthoate, diphenyl carbonate, glycol carbonate, dibenzyl terephthalate, dimethyl terephthalate, 1,4-dimethoxynaphthalene, 1,4-diethoxynaphthalene, 1,4-dibenzyloxynaphthalene, 1,2-diphenoxyethane, 1,2-bis(3-methylphenoxy)ethane, 1,2-bis(4-methylphenoxy)ethane, 1,4-diphenoxy-2-butene, 1,2-bis(4-methoxyphenylthio)ethane, dibenzoyl methane, 1,4-diphenylthiobutane, 1,4-diphenylthio-2-butene, 1,3-bis(2-vinyloxyethoxy)benzene, 1,4-bis(2-vinyloxyethoxy)benzene, p-(2-vinyloxyethoxy)biphenyl, p-aryloxybiphenyl, p-propargyloxybiphenyl, dibenzoyloxymethane, dibenzoyloxypropane, dibenzyl disulfide, 1,1-diphenyl ethanol, 1,1-diphenyl propanol, p-benzyloxybenzyl alcohol, 1,3-phenoxy-2-propanol, N-octadecyl carbamoyl-p-methoxycarbonyl benzene, N-octadecyl carbamoyl benzene, 1,2-bis(4-methoxyphenoxy)propane, 1,5-bis(4-methoxyphenoxy)-3-oxapentane, dibenzyl oxalate, bis(4-methylbenzyl) oxalate, and bis(4-chlorobenzyl) oxalate. One of these thermally fusible substances may be used alone or two or more of these thermally fusible substances may be used in combination.

As needed, various hindered phenol compounds or hindered amine compounds that have an electron-accepting property but have a relatively low color developing ability may be added in the thermosensitive recording layer as auxiliary additives. Specific examples of such auxiliary additives include 2,2′-methylene bis(4-ethyl-6-tertiary butylphenol), 4,4′-butylidene bis(6-tertiary butyl-2-methylphenol), 1,1,3-tris(2-methyl-4-hydroxy-5-tertiary butylphenyl)butane, 1,1,3-tris(2-methyl-4-hydroxy-5-cyclohexylphenyl)butane, 4,4′-thio bis(6-tertiary butyl-2-methylphenol), tetrabromobisphenol A, tetrabromobisphenol S, 4,4-thio bis(2-methylphenol), 4,4′-thio bis(2-chlorophenol), tetrakis(1,2,2,6,6-pentamethyl-4-piperidyl)-1,2,3,4-butane tetracarboxylate, and tetrakis(1,2,2,6,6-tetramethyl-4-piperidyl)-1,2,3,4-butane tetracarboxylate.

—Other Components—

The other components are not particularly limited and may be appropriately selected depending on the intended purpose. Examples of the other components include a surfactant, a lubricant, and a loading material.

Examples of the lubricant include higher fatty acids or metal salts of higher fatty acids, higher fatty acid amides, higher fatty acid esters, animal waxes, plant waxes, mineral waxes, and petroleum waxes.

Examples of the loading material include: inorganic powders of, for example, calcium carbonate, silica, zinc oxide, titanium oxide, aluminum hydroxide, zinc hydroxide, barium sulfate, clay, kaolin, talc, surface-treated calcium, and surface-treated silica; and organic powders of, urea-formalin resins, styrene-methacrylic acid copolymers, polystyrene resins, and vinylidene chloride resins.

The thermosensitive recording layer can be formed by a publicly known method that is not particularly limited. For example, the thermosensitive recording layer can be formed by subjecting the leuco dye and the developer together with the binder resin and the other components to pulverization and dispersion using a disperser such as a ball mill, an attritor, and a sand mill until the dispersed particle diameter becomes 0.1 micrometers or greater but 3 micrometers or less, mixing the resultant with, for example, the loading material and a dispersion liquid of the thermally fusible substance as needed, to prepare a thermosensitive recording layer coating liquid, coating the thermosensitive recording layer coating liquid on an undercoat layer, and drying the thermosensitive recording layer coating liquid.

The coating method is not particularly limited and may be appropriately selected depending on the intended purpose. Examples of the coating method include a blade coating method, a gravure coating method, a gravure offset coating method, a bar coating method, a roll coating method a knife coating method, an air knife coating method, a comma coating method, a U-comma coating method, an AKKU coating method, a smoothing coating method, a microgravure coating method, a reverse roll coating method, a 4- or 5-roll coating method, a dip coating method, a curtain coating method, a slide coating method, and a die coating method.

The dry amount of the thermosensitive recording layer attached is not particularly limited, may be appropriately selected depending on the intended purpose, and is preferably 1 g/m² or greater but 20 g/m² or less and more preferably 3 g/m² or greater but 10 g/m² or less.

<Support>

The shape, structure, size, and material of the support are not particularly limited and may be appropriately selected depending on the intended purpose. Examples of the shape of the support include a flat plate shape and a sheet shape. Examples of the structure of the support include a single-layered structure and a laminated structure. The size of the support may be appropriately selected depending on, for example, the size of the thermosensitive recording medium.

The material of the support is not particularly limited and may be appropriately selected depending on the intended purpose. Examples of the material of the support include inorganic materials and organic materials.

Examples of the inorganic materials include glass, quartz, silicon, silicon oxide, aluminum oxide, SiO₂, and metals.

Examples of the organic materials include: paper such as pure paper, art paper, coat paper, and synthetic paper; cellulose derivatives such as cellulose triacetate; and plastic films of, for example, polyester resins such as polyethylene terephthalate (PET) and polybutylene terephthalate, polycarbonate, polystyrene, polymethyl methacrylate, polyethylene, and polypropylene. One of these materials may be used alone or two or more of these materials may be used in combination.

In order to improve adhesiveness of the undercoat layer with the support, it is preferable to subject the support to surface reformation by, for example, corona discharge treatment, oxidation reaction treatment (e.g., chromic acid), etching treatment, treatment for imparting easy adhesiveness, and antistatic treatment. It is preferable to make the support white by adding, for example, a white pigment such as titanium oxide.

The average thickness of the support is not particularly limited, may be appropriately selected depending on the intended purpose, and is preferably 20 micrometers or greater but 2,000 micrometers or less and more preferably 50 micrometers or greater but 1,000 micrometers or less.

<Undercoat Layer>

The undercoat layer contains hollow particles, preferably contains a resin and a cross-linking agent, and further contains other components as needed. The undercoat layer may also be referred to as intermediate layer.

—Hollow Particles—

The hollow particles are not particularly limited and may be appropriately selected depending on the intended purpose. The ratio of hollowness of the hollow particles is preferably 50% or greater and more preferably 80% or greater. When the ratio of hollowness of the hollow particles is 50% or greater, color developing sensitivity and color developing accuracy are good.

The ratio of hollowness is the ratio between the volume of a hollow particle and a void and expressed in percentage (%). As the hollow particles can be seen as almost spherical, the ratio of hollowness is represented by the following formula.

Ratio of hollowness (%)=[(volume of void)/(volume of hollow particle)]×100

The hollow particles are nonfoamable hollow particles that are already in a foamed state and contain a thermoplastic resin shell and air or any other gas inside the shell. The volume average particle diameter of the hollow particles is preferably 0.4 micrometers or greater but 10 micrometers or less. When the volume average particle diameter (outer particle diameter) is 0.4 micrometers or greater but 10 micrometers or less, a good sensitivity improving effect can be obtained. Hence, it is preferable that the hollow particles have a volume average particle diameter in the range described above and a uniform distribution peak with a small variation.

Examples of the thermoplastic resin forming the shell of the hollow particles include polystyrene resins, polyvinyl chloride resins, polyvinylidene chloride resins, polyvinyl acetate resins, polyacrylic acid ester resins, polyacrylonitrile resins, polybutadiene resins, and copolymers of these resins. Among these thermoplastic resins, copolymers mainly formed of vinylidene chloride and acrylonitrile are particularly preferable.

The hollow particles may be appropriately produced products or commercially available products. Examples of the commercially available products include hollow particles (available from Rohm and Haas Company, ROPAQUE SN-1055, with a ratio of hollowness of 50%), hollow particles (available from Rohm and Haas Company, ROPAQUE OP-62, with a ratio of hollowness of 33%), hollow particles (available from Matsumoto Yushi-Seiyaku Co., Ltd., MATSUMOTO MICROSPHERE R-500, with a ratio of hollowness of 90%), and hollow particles (available from JSR Corporation, SX8782(D), with a ratio of hollowness of 50%).

The content of the hollow particles in the undercoat layer is not particularly limited, may be appropriately selected depending on the intended purpose, and is preferably 20% by mass or greater but 50% by mass or less and more preferably 30% by mass or greater but 40% by mass or less.

—Binder Resin—

The binder resin is not particularly limited, may be appropriately selected depending on the intended purpose, and is preferably any one of a water-soluble polymer and an aqueous polymer emulsion.

The water-soluble polymer is not particularly limited and may be appropriately selected depending on the intended purpose. Examples of the water-soluble polymer include: polyvinyl alcohols; modified polyvinyl alcohols such as polyvinyl alcohols containing a carboxyl group; starch or derivatives of starch; cellulose derivatives such as methoxy cellulose, hydroxyethyl cellulose, carboxymethyl cellulose, methyl cellulose, and ethyl cellulose; and polyurethane, sodium polyacrylate, polyvinyl pyrrolidone, acrylamide/acrylic acid ester copolymers, acrylamide/acrylic acid ester/methacrylic acid terpolymers, alkali salts of styrene/maleic anhydride copolymers, alkali salts of isobutylene/maleic anhydride copolymers, polyacrylamide, sodium alginate, gelatin, and casein. One of these water-soluble polymers may be used alone or two or more of these water-soluble polymers may be used in combination.

The aqueous polymer emulsion is not particularly limited and may be appropriately selected depending on the intended purpose. Examples of the aqueous polymer emulsion include: latexes of, for example, acrylic resins, modified acrylic resins such as acrylic resins containing a carboxyl group, styrene/butadiene copolymers, and styrene/butadiene/acrylic-based copolymers; and emulsions of, for example, vinyl acetate resins, vinyl acetate/acrylic acid copolymers, styrene/acrylic acid ester copolymers, acrylic acid ester resins, and polyurethane resins. One of these aqueous polymer emulsions may be used alone or two or more of these aqueous polymer emulsions may be used in combination.

The content of the binder resin in the undercoat layer is not particularly limited, may be appropriately selected depending on the intended purpose, and is preferably 30 parts by mass or greater but 300 parts by mass or less and more preferably 40 parts by mass or greater but 200 parts by mass or less relative to 100 parts by mass of the hollow particles.

When the content of the binder resin is 30 parts by mass or greater but 300 parts by mass or less, a sufficient binding force can be obtained between the support and the undercoat layer. This provides a good color developing property.

—Cross-Linking Agent—

The cross-linking agent is not particularly limited and may be appropriately selected depending on the intended purpose. Examples of the cross-linking agent include compounds containing an oxazoline group, glyoxal derivatives, methylol derivatives, epichlorohydrin derivatives, epoxy compounds, aziridine compounds, hydrazine, hydrazide derivatives, and carbodiimide derivatives. One of these cross-linking agents may be used alone or two or more of these cross-linking agents may be used in combination. Among these cross-linking agents, compounds containing an oxazoline group are particularly preferable because these compounds have a high bindability with the support and water resistance to qualify as the undercoat layer, and needs a short curing time for obtaining water resistance.

The compounds containing an oxazoline group may be appropriately synthesized products or commercially available products. Examples of the commercially available products include EPOCROS WS-700 (available from Nippon Shokubai Co., Ltd.).

The content of the compound containing an oxazoline group is not particularly limited, may be appropriately selected depending on the intended purpose, and is preferably 20 parts by mass or greater but 70 parts by mass or less and more preferably 30 parts by mass or greater but 50 parts by mass or less relative to 100 parts by mass of the binder resin. It is preferable that the content of the compound containing an oxazoline group be 20 parts by mass or greater but 70 parts by mass or less, because a long curing time is not needed for making the undercoat layer water-resistant, and a good color developing property is obtained.

—Other Components—

The other components are not particularly limited and may be appropriately selected depending on the intended purpose. Examples of the other components include a surfactant, a filler, a lubricant, and a loading material.

The method for forming the undercoat layer is not particularly limited and may be appropriately selected depending on the intended purpose. For example, the undercoat layer can be formed by subjecting the binder resin, the hollow particles, and water, preferably, the cross-linking agent, and as needed, the other components to dispersion using a disperser to prepare an undercoat layer coating liquid, coating the undercoat layer coating liquid on the support, and drying the undercoat layer coating liquid.

The coating method is not particularly limited and may be appropriately selected depending on the intended purpose. Examples of the coating method include a blade coating method, a gravure coating method, a gravure offset coating method, a bar coating method, a roll coating method, a knife coating method, an air knife coating method, a comma coating method, a U-comma coating method, an AKKU coating method, a smoothing coating method, a microgravure coating method, a reverse roll coating method, a 4- or 5-roll coating method, a dip coating method, a curtain coating method, a slide coating method, and a die coating method.

The dry amount of the undercoat layer attached is not particularly limited, may be appropriately selected depending on the intended purpose, and is preferably 1 g/m² or greater but 5 g/m² or less and more preferably 2 g/m² or greater but 5 g/m² or less.

—Protective Layer—

The protective layer contains a binder resin and a cross-linking agent, and further contains other components as needed.

The binder resin is not particularly limited, may be appropriately selected depending on the intended purpose, and is particularly preferably a water-soluble resin.

Examples of the water-soluble resin include polyvinyl alcohols, modified polyvinyl alcohols, starch or derivatives of starch, cellulose derivatives such as methoxy cellulose, hydroxyethyl cellulose, carboxymethyl cellulose, methyl cellulose, and ethyl cellulose, sodium polyacrylate, polyvinyl pyrrolidone, acrylamide-acrylic acid ester copolymers, acrylamide-acrylic acid ester-methacrylic acid terpolymers, alkali salts of styrene-maleic anhydride copolymers, alkali salts of isobutylene-maleic anhydride copolymers, polyacrylamide, modified polyacrylamide, methylvinyl ether-maleic anhydride copolymers, carboxy-modified polyethylene, polyvinyl alcohol-acrylamide block copolymers, melamine-formaldehyde resins, urea-formaldehyde resins, sodium alginate, gelatin, and casein. One of these water-soluble resins may be used alone or two or more of these water-soluble resins may be used in combination. Among these water-soluble resins, modified polyvinyl alcohols are preferable.

Examples of the modified polyvinyl alcohols include: diacetone-modified polyvinyl alcohols; acetoacetyl-modified polyvinyl alcohols; and carboxylic acid-modified polyvinyl alcohols such as itaconic acid-modified polyvinyl alcohols and maleic acid-modified polyvinyl alcohols.

The cross-linking agent is not particularly limited and may be appropriately selected depending on the intended purpose so long as the cross-linking agent can reduce water solubility of the water-soluble resin by undergoing a reaction with the water-soluble resin. Examples of the cross-linking agent include glyoxal derivatives, methylol derivatives, epichlorohydrin, polyamide epichlorohydrin, epoxy compounds, aziridine compounds, hydrazine, hydrazide derivatives, oxazoline derivatives, and carbodiimide derivatives. One of these cross-linking agents may be used alone or two or more of these cross-linking agents may be used in combination. Among these cross-linking agents, polyamide epichlorohydrin is particularly preferable because polyamide epichlorohydrin is highly safe in handling and needs a short curing time for obtaining water resistance.

The content of the polyamide epichlorohydrin is not particularly limited, may be appropriately selected depending on the intended purpose, and is preferably 10 parts by mass or greater but 60 parts by mass or less and more preferably 20 parts by mass or greater but 50 parts by mass or less relative to 100 parts by mass of the binder resin.

It is preferable to add a pigment (filler) in the protective layer as needed. Examples of the pigment to be used in the protective layer include inorganic pigments such as zinc oxide, calcium carbonate, barium sulfate, titanium oxide, lithopone, talc, pyrophyllite, kaolin, aluminum hydroxide, and calcined kaolin, and organic pigments such as cross-linked polystyrene resins, urea resins, silicone resins, cross-linked polymethyl methacrylate resins, and melamine-formaldehyde resins.

It is possible to use auxiliary additive components hitherto used, such as a surfactant, a heat-fusible substance, a lubricant, and a pressure coloring inhibitor in the protective layer in combination with the resin, a water resistant additive, and the pigment described above.

The protective layer can be formed by a publicly known method that is not particularly limited.

The average thickness of the protective layer is not particularly limited, may be appropriately selected depending on the intended purpose, and is preferably 0.5 micrometers or greater but 5 micrometers or less and more preferably 1 micrometer or greater but 3 micrometers or less.

<Other Layers>

The other layers are not particularly limited and may be appropriately selected depending on the intended purpose. Examples of the other layers include a back layer.

—Back Layer—

As needed, the back layer may be provided on a surface of the support at a side at which the thermosensitive recording layer is not provided.

The back layer contains a filler and a binder resin and further contains other components such as a lubricant and a coloring pigment as needed.

Usable examples of the filler include inorganic fillers and organic fillers.

Examples of the inorganic fillers include carbonates, silicates, metal oxides, and sulfuric acid compounds.

Examples of the organic fillers include silicone resins, cellulose, epoxy resins, nylon resins, phenol resins, polyurethane resins, urea resins, melamine resins, polyester resins, polycarbonate resins, styrene resins, acrylic resins, polyethylene resins, formaldehyde resins, and polymethyl methacrylate resins.

The binder resin is not particularly limited, may be appropriately selected depending on the intended purpose, and may be the same as the binder resin in the thermosensitive recording layer.

The average thickness of the back layer is not particularly limited, may be appropriately selected depending on the intended purpose, and is preferably 0.1 micrometers or greater but 20 micrometers or less and more preferably 0.3 micrometers or greater but 10 micrometers or less.

The embodiment of the thermosensitive recording medium of the present invention is not particularly limited and may be appropriately selected depending on the intended purpose. For example, the thermosensitive recording medium may be used as is as a label, or may have on the protective layer or the support, a layer on which information such as characters, marks, pictures, and two-dimensional codes such as barcodes or QR codes (registered trademark) is recorded. The thermosensitive recording medium may be an embodiment in which a viscous layer is provided at a side of the support opposite to the side at which the thermosensitive recording layer is provided.

The shape of the thermosensitive recording medium of the present invention is not particularly limited and may be appropriately selected depending on the intended purpose. Examples of the shape of the thermosensitive recording medium include a label shape, a sheet shape, and a roll shape.

Specific examples of the embodiment of the thermosensitive recording medium of the present invention will be described below.

<Thermosensitive Recording Label>

The thermosensitive recording medium of the present embodiment is a thermosensitive recording label that includes a viscous layer on a surface of the support opposite to the surface provided with the thermosensitive recording layer, and a release paper on the viscous layer, and further includes other layers as needed. The viscous layer may be coated all over the label or may be coated only on a portion.

The material of the viscous layer is not particularly limited and may be appropriately selected depending on the intended purpose. Examples of the material of the adhesive layer include urea resins, melamine resins, phenol resins, epoxy resins, vinyl acetate resins, vinyl acetate-acrylic copolymers, ethylene-vinyl acetate copolymers, acrylic resins, polyvinyl ether resins, vinyl chloride-vinyl acetate copolymers, polystyrene resins, polyester resins, polyurethane resins, polyamide resins, chlorinated polyolefin resins, polyvinyl butyral resins, acrylic acid ester copolymers, methacrylic acid ester copolymers, natural rubbers, cyanoacrylate resins, and silicone resins. One of these materials may be used alone or two or more of these materials may be used in combination.

<Linerless Thermosensitive Recording Medium>

(Type with a Release Layer)

The thermosensitive recording medium of the present embodiment is a linerless thermosensitive recording medium that further includes a release layer as the topmost layer on the surface (front surface) of the support provided with the thermosensitive recording layer, and further includes a viscous layer on the surface (back surface) of the support opposite to the surface provided with the thermosensitive recording layer. The thermosensitive recording medium may further include other layers as needed.

The release layer is preferably a layer formed of a material having a good separability from the viscous layer, and is particularly preferably a layer containing silicone.

The linerless thermosensitive recording medium of the present embodiment can be handled in a roll form wound in a manner that the viscous layer is stacked on the release layer.

(Type without a Release Layer)

The thermosensitive recording medium of the present embodiment is a linerless thermosensitive recording medium that further includes a viscous layer on a surface of the support opposite to the surface provided with the thermosensitive recording layer and further includes other layers as needed. The viscous layer is a thermosensitive viscous layer that expresses a viscous property when heated.

The thermosensitive viscous layer contains a thermoplastic resin and a heat-fusible substance, and further contains a tackifier as needed.

The thermoplastic resin imparts a viscous force and an adhesive force. The heat-fusible substance is a solid at normal temperature and thus not imparts plasticity to a resin, but melts when heated to swell or soften a resin to make the resin express a viscous property. The tackifier has a function of improving a viscous property.

<Thermosensitive Magnetic Recording Paper>

The thermosensitive recording medium of the present embodiment is a thermosensitive magnetic recording paper that includes a magnetic recording layer on a surface of the support opposite to the surface provided with the thermosensitive recording layer, and further includes other layers as needed.

The magnetic recording layer is formed by coating of, for example, iron oxide and barium ferrite, and, for example, a vinyl chloride resin, a urethane resin, and a nylon resin on the support, or by such a method as, for example, vapor deposition or sputtering without the use of a resin. It is preferable to provide the magnetic recording layer on a surface of the support opposite to the surface provided with the thermosensitive recording layer, but it is also possible to provide the magnetic recording layer between the support and the thermosensitive recording layer or on at least a part of the thermosensitive recording layer.

(Recording Method)

A recording method using the thermosensitive recording medium of the present invention is not particularly limited and may be appropriately selected depending on the intended purpose. Examples of the recording method include a thermal head and a laser.

The shape, structure, and size of the thermal head are not particularly limited and may be appropriately selected depending on the intended purpose.

The laser is not particularly limited and may be appropriately selected depending on the intended purpose. Examples of the laser include a CO₂ laser and a semiconductor laser having a wavelength of from 9.3 micrometers or longer but 10.6 micrometers or shorter.

(Applications)

The thermosensitive recording medium of the present invention has a high color developing sensitivity, a high image density, and an excellent hand cream resistance. Therefore, the thermosensitive recording medium of the present invention can be used in many fields such as: the field of POS for, for example, perishable foods, box lunches, and delicatessen; the field of copying of, for example, books and documents; the field of communication by, for example, facsimile; the field of ticketing of, for example, receipts and signed receipts by ticketing machines; baggage tags in the airline industry; and pill cases and pill bottles.

(Article)

An article of the present invention includes the thermosensitive recording medium of the present invention.

As the thermosensitive recording medium, the thermosensitive recording medium of the present invention can be favorably used.

What is meant when it is said that an article includes the thermosensitive recording medium of the present invention is a state that the thermosensitive recording medium of the present invention is, for example, pasted or attached on the article.

The article of the present invention is not particularly limited and may be appropriately selected depending on the intended purpose so long as the article includes the thermosensitive recording medium of the present invention. Examples of the article include packing materials, packaging materials, and wrapping paper. More specific examples of the article include packaging materials for perishable foods, box lunches, delicatessen, books, and documents.

### EXAMPLES

The present invention will be described below by way of Examples. The present invention should not be construed as being limited to these Examples.

### Example 1

<Production of Thermosensitive Recording Medium>

—Preparation of Undercoat Layer Coating Liquid <A liquid>—

The components in the composition described below were mixed and stirred, to prepare an undercoat layer coating liquid <A liquid>.

<Composition of a Liquid>
Hollow particles (with a ratio of hollowness of 90%, a volume average particle diameter of 4.4 micrometers, and a solid concentration of 33% by mass): 20 parts by mass
Styrene/butadiene copolymer latex (with a solid concentration of 47.5% by mass): 20 parts by mass
10% by mass polyvinyl alcohol aqueous solution (available from Kuraray Co., Ltd., PVA117): 20 parts by mass
Ion-exchanged water: 40 parts by mass

—Preparation of Thermosensitive Recording Layer Coating Liquid <E liquid>—

The compositions of <B liquid>, <C liquid>, and <D liquid>, each of which includes components described below, were prepared in a manner that a leuco dye or a developer was dispersed using a sand grinder so that the average particle diameters of the leuco dye or the developer in any of <B liquid>, <C liquid>, and <D liquid> became 0.5 micrometers, 1.0 micrometer, and 1.0 micrometer, respectively. Next, the <B liquid>(20 parts by mass), the <C liquid>(30 parts by mass), the <D liquid>(10 parts by mass), a styrene-butadiene copolymer latex (with a solid concentration of 47.5% by mass) (5 parts by mass), a 10% by mass itaconic acid-modified polyvinyl alcohol aqueous solution (10 parts by mass), and ion-exchanged water (40 parts by mass) were mixed and stirred, to prepare a thermosensitive recording layer coating liquid <E liquid>.

<Composition of B Liquid>
Leuco dye (3-dibutylamino-6-methyl-7-anilinofluoran): 20 parts by mass
10% by mass itaconic acid-modified polyvinyl alcohol aqueous solution (available from Kuraray Co., Ltd., 25-88KL): 40 parts by mass
Surfactant (available from Nippon Nyukazai Co., Ltd., NEWCOL 290, with a solid concentration of 100% by mass): 0.2 parts by mass
Ion-exchanged water: 40 parts by mass

<Composition of C Liquid>
4-Hydroxy-4′-benzyloxydiphenylsulfone: 20 parts by mass
10% by mass itaconic acid-modified polyvinyl alcohol aqueous solution (available from Kuraray Co., Ltd., 25-88KL): 20 parts by mass
Amorphous silica (available from Mizusawa Industrial Chemicals, Ltd., MIZUKASIL P527): 15 parts by mass
Surfactant (available from Nissin Chemical Co., Ltd., PD-001, with a solid concentration of 100% by mass): 0.2 parts by mass
Ion-exchanged water: 60 parts by mass

<Composition of D Liquid>
D90 (available from Nippon Soda Co., Ltd.) represented by the following structural formula A: 20 parts by mass

<Structural Formula A>

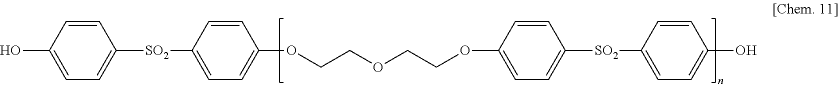

D90 is a mixture of compounds represented by structural formula A where n is a numerical value of from 1 to 11.
10% by mass itaconic acid-modified polyvinyl alcohol aqueous solution (available from Kuraray Co., Ltd., 25-88KL): 20 parts by mass
Amorphous silica (available from Mizusawa Industrial Chemicals, Ltd., MIZUKASIL P527): 15 parts by mass
Surfactant (available from Nissin Chemical Co., Ltd., PD-001, with a solid concentration of 100% by mass): 0.2 parts by mass
Ion-exchanged water: 60 parts by mass

—Preparation of Protective Layer Coating Liquid <G Liquid>—

A mixture having the composition described below was subjected to dispersion using a sand grinder such that the volume average particle diameter became 0.5 micrometers, to prepare <F liquid>.

<Composition of F Liquid>
Aluminum hydroxide: 30 parts by mass
10% by mass itaconic acid-modified polyvinyl alcohol aqueous solution (available from Kuraray Co., Ltd., 25-88KL): 30 parts by mass
Ion-exchanged water: 40 parts by mass

Next, the components in the composition described below were mixed and stirred, to prepare a protective layer coating liquid <G liquid>.

<Composition of G liquid>
<F liquid>described above: 30 parts by mass
10% by mass diacetone-modified polyvinyl alcohol aqueous solution (available from Japan VAM & Poval Co., Ltd., DF-17): 50 parts by mass
Cross-linking agent liquid (adipic acid dihydrazide, with a solid concentration of 10% by mass): 20 parts by mass
Montanic acid ester wax dispersion liquid (with a solid concentration of 30% by mass): 5 parts by mass
Ion-exchanged water: 15 parts by mass

—Preparation of Protective Layer Coating Liquid <H Liquid>—

Next, the components in the composition described below were mixed and stirred, to prepare a protective layer coating liquid <H liquid>.

<Composition of H Liquid>
<F liquid>described above: 30 parts by mass
10% by mass itaconic acid-modified polyvinyl alcohol aqueous solution (available from Kuraray Co., Ltd., 25-88KL): 50 parts by mass
Cross-linking agent liquid (polyamide epichlorohydrin, with a solid concentration of 10% by mass): 20 parts by mass
Montanic acid ester wax dispersion liquid (with a solid concentration of 30% by mass): 5 parts by mass
Ion-exchanged water: 15 parts by mass

—Preparation of Protective Layer Coating Liquid <I Liquid>—

Next, the components in the composition described below were mixed and stirred, to prepare a protective layer coating liquid <I liquid>.

<Composition of I Liquid>
<F liquid>described above: 30 parts by mass
10% by mass completely saponified polyvinyl alcohol aqueous solution (available from Kuraray Co., Ltd., 28-98): 50 parts by mass
Oxazoline-based cross-linking agent liquid (available from Nippon Shokubai Co., Ltd., WS-500, with a solid concentration of 39% by mass): 5 parts by mass
Montanic acid ester wax dispersion liquid (with a solid concentration of 30% by mass): 5 parts by mass
Ion-exchanged water: 15 parts by mass

—Preparation of Protective Layer Coating Liquid <J Liquid>—

Next, the components in the composition described below were mixed and stirred, to prepare a protective layer coating liquid <J liquid>.

<Composition of J Liquid>
<F liquid>described above: 30 parts by mass
Core-shell type acrylic emulsion (available from Mitsui Chemicals, Inc., BARIASTAR B-2000, with a solid concentration of 19% by mass): 27 parts by mass
Cross-linking agent liquid (polyamide epichlorohydrin, with a solid concentration of 10% by mass): 20 parts by mass
Montanic acid ester wax dispersion liquid (with a solid concentration of 30% by mass): 5 parts by mass
Ion-exchanged water: 15 parts by mass

—Preparation of Protective Layer Coating Liquid <K Liquid>—

Next, the components in the composition described below were mixed and stirred, to prepare a protective layer coating liquid <K liquid>.

<Composition of K Liquid>
<F liquid>described above: 30 parts by mass
10% by mass acetoacetyl-modified polyvinyl alcohol aqueous solution (available from Nippon Synthetic Chemical Industry Co., Ltd., Z-200): 50 parts by mass
Cross-linking agent liquid (adipic acid dihydrazide, with a solid concentration of 10% by mass): 20 parts by mass
Montanic acid ester wax dispersion liquid (with a solid concentration of 30% by mass): 5 parts by mass
Ion-exchanged water: 15 parts by mass

Next, the undercoat layer coating liquid <A liquid>, the thermosensitive recording layer coating liquid <E liquid>, and the protective layer coating liquid <G liquid> were coated on the surface of a support, which was paper having a basis weight of 62 g/m², and dried such that the dry amounts attached as an undercoat layer, a thermosensitive recording layer, and a protective layer became 3.0 g/m², 2.5 g/m², and 3.0 g/m².

Next, the resultant was subjected to surface treatment using a super-calender such that the surface smoothness became from 1,500 seconds through 2,500 second, then hermetically sealed in a high density polyethylene bag, and cured in an environment of 40 degrees C. for a predetermined period. In this way, a thermosensitive recording medium of Example 1 was produced.

### Examples 2 to 15 and Comparative Examples 1 to 7

Thermosensitive recording media of Examples 2 to 15 and Comparative Examples 1 to 7 were produced in the same manner as in Example 1, except that the combination of the thermosensitive recording layer and the protective layer of Example 1 was changed to the combinations presented in Table 1 below.

TABLE 1

| | Heat-sensitive print layer | | | | |
|---|---|---|---|---|---|
| | <C liquid> | | | | Protective |
| | (part by mass) | | <D liquid> | | layer |
| | | Content ratio of | (part by mass) | | Protective |
| | Content | 4-hydroxy-4′- | Content | Content | layer |
| | of <C | benzyloxydiphenyl | of <D | ratio of | coating |
| | liquid> | sulfone | liquid> | D 90 | liquid |
| Ex. 1 | 30.0 | 1.0 | 10.0 | 0.3 | G liquid |
| Ex. 2 | 38.5 | 1.0 | 1.5 | 0.04 | G liquid |
| Ex. 3 | 36.4 | 1.0 | 3.6 | 0.1 | G liquid |
| Ex. 4 | 26.7 | 1.0 | 13.3 | 0.5 | G liquid |
| Ex. 5 | 22.2 | 1.0 | 17.8 | 0.8 | G liquid |
| Ex. 6 | 20.0 | 1.0 | 20.0 | 1.0 | G liquid |
| Ex. 7 | 18.2 | 1.0 | 21.8 | 1.2 | G liquid |
| Ex. 8 | 16.0 | 1.0 | 24.0 | 1.5 | G liquid |
| Ex. 9 | 13.3 | 1.0 | 26.7 | 2.0 | G liquid |
| Ex. 10 | 11.4 | 1.0 | 28.6 | 2.5 | G liquid |
| Ex. 11 | 10.5 | 1.0 | 29.5 | 2.8 | G liquid |
| Ex. 12 | 30.0 | 1.0 | 10.0 | 0.3 | K liquid |
| Ex. 13 | 30.0 | 1.0 | 10.0 | 0.3 | H liquid |
| Ex. 14 | 30.0 | 1.0 | 10.0 | 0.3 | I liquid |
| Ex. 15 | 30.0 | 1.0 | 10.0 | 0.3 | J liquid |
| Comp. | 10.0 | 1.0 | 30.0 | 3.0 | G liquid |
| Ex. 1 | | | | | |
| Comp. | 6.7 | 1.0 | 33.3 | 5.2 | G liquid |
| Ex. 2 | | | | | |
| Comp. | 40.0 | 1.0 | 0.0 | 0.0 | G liquid |
| Ex. 3 | | | | | |
| Comp. | 6.7 | 1.0 | 33.3 | 5.2 | I liquid |
| Ex. 4 | | | | | |
| Comp. | 6.7 | 1.0 | 33.3 | 5.2 | J liquid |
| Ex. 5 | | | | | |
| Comp. | 40.0 | 1.0 | 0.0 | 0.0 | I liquid |
| Ex. 6 | | | | | |
| Comp. | 40.0 | 1.0 | 0.0 | 0.0 | J liquid |
| Ex. 7 | | | | | |

Next, various properties of the produced thermosensitive recording media of Examples 1 to 15 and Comparative Examples 1 to 7 were evaluated in the manners described below. The results are presented in Table 2.

<Maximum Color Developing Density>

With a thermal gradient tester (HEAT GRADIENT, available from Toyo Seiki Co., Ltd.), each thermosensitive recording medium was caused to develop a color at 0.2 MPa for 2 seconds at each of 130 degrees C., 140 degrees C., 150 degrees C., 160 degrees C., and 170 degrees C., and the resulting image densities were measured with a Macbeth reflection densitometer (RD-914, available from Gretag Macbeth Ltd.). The maximum density was recorded and evaluated according to the criteria described below.

<Evaluation Criteria>

A: The maximum color developing density was 1.30 or greater.

B: The maximum color developing density was 1.20 or greater but 1.29 or less.

C: The maximum color developing density was 1.19 or less.

<Sensitivity Magnification>

With a recording simulator (available from Ohkura Denki), recording was performed on each thermosensitive recording medium with a pulse width (applied energy) of from 0.1 msec through 1.2 msec at 0.1 msec intervals under a head power of 0.45 w/dot, and the image density was measured with a Macbeth reflection densitometer (RD-914, available from Gretag Macbeth Ltd.). The pulse width needed to obtain an image density of 1.0 was calculated. Based on the obtained energy value, a sensitivity magnification relative to Example 1 was calculated according to the mathematical formula described below. The sensitivity magnification was evaluated according to the criteria described below.

Sensitivity magnification=(Pulse width in Example 1)/(Pulse width for measured sample)

<Evaluation Criteria>

A: The sensitivity magnification was 0.95 or greater.

B: The sensitivity magnification was 0.75 or greater but 0.94 or less.

C: The sensitivity magnification was 0.74 or less.

<Hand Cream Resistance>

With a recording simulator (available from Ohkura Denki), a sample was produced by performing recording on each thermosensitive recording medium with a pulse width (applied energy) of 1.0 msec at 0.1 msec intervals under a head power of 0.45 w/dot.

A hand cream (available from Curel Advanced Ceramide, THERAPY DAILY MOISTURE) was uniformly applied on the image portion of the produced sample with a cotton cloth and stored at 40 degrees C. at 90% RH for 15 hours. The image density after the storage was measured with a Macbeth reflection densitometer (RD-914, available from Gretag Macbeth Ltd.), and an image remaining ratio was calculated according to the formula described below and evaluated according to the criteria described below.

Image remaining ratio (%)=[(Image density after hand cream application and storage for 15 hours)/(Image density before hand cream application)]×100

<Evaluation Criteria>

A: The image remaining ratio was 80% or greater.

B: The image remaining ratio was 60% or greater but 79% or less.

C: The image remaining ratio was 59% or less

TABLE 2

| | Maximum color | | | | Hand cream | |
|---|---|---|---|---|---|---|
| | developing | | Sensitivity | | resistance | |
| | density | | magnification | | Image | |
| | Measured | Eval- | Calculated | Eval- | remaining | Eval- |
| | value | uation | value | uation | ratio (%) | uation |
| Ex. 1 | 1.36 | A | 1.00 | A | 83 | A |
| Ex. 2 | 1.42 | A | 1.08 | A | 69 | B |
| Ex. 3 | 1.39 | A | 1.05 | A | 71 | B |
| Ex. 4 | 1.34 | A | 0.99 | A | 85 | A |
| Ex. 5 | 1.33 | A | 0.98 | A | 88 | A |
| Ex. 6 | 1.32 | A | 0.96 | A | 94 | A |
| Ex. 7 | 1.31 | A | 0.94 | B | 95 | A |
| Ex. 8 | 1.28 | B | 0.92 | B | 97 | A |
| Ex. 9 | 1.27 | B | 0.89 | B | 97 | A |
| Ex. 10 | 1.25 | B | 0.83 | B | 100 | A |
| Ex. 11 | 1.21 | B | 0.79 | B | 100 | A |
| Ex. 12 | 1.36 | A | 1.00 | A | 83 | A |
| Ex. 13 | 1.37 | A | 1.01 | A | 81 | A |
| Ex. 14 | 1.36 | A | 1.02 | A | 70 | B |
| Ex. 15 | 1.38 | A | 1.02 | A | 75 | B |
| Comp. Ex. 1 | 1.18 | C | 0.70 | C | 100 | A |
| Comp. Ex. 2 | 1.08 | C | 0.69 | C | 100 | A |
| Comp. Ex. 3 | 1.40 | A | 1.11 | A | 42 | C |
| Comp. Ex. 4 | 1.09 | C | 0.72 | C | 99 | A |
| Comp. Ex. 5 | 1.10 | C | 0.70 | C | 99 | A |
| Comp. Ex. 6 | 1.39 | A | 1.11 | A | 44 | C |
| Comp. Ex. 7 | 1.42 | A | 1.10 | A | 41 | C |

Aspects of the present invention are as follows, for example.

<1> A thermosensitive recording medium including:

a support; and

a thermosensitive recording layer provided over at least one surface of the support and containing a leuco dye, a first developer, and a second developer,

wherein the first developer and the second developer are 4-hydroxy-4′-benzyloxydiphenylsulfone and a diphenylsulfone compound represented by the following general formula (1), respectively,

wherein a content of the diphenylsulfone compound represented by general formula (1) is less than 3.0 parts by mass relative to 1.0 part by mass of the 4-hydroxy-4′-benzyloxydiphenylsulfone,

<General Formula (1)>

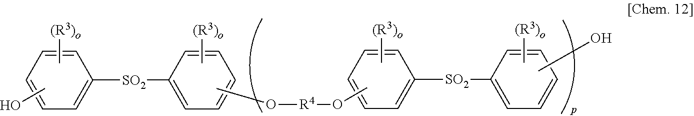

where in general formula (1), R³ represents any one of a hydrogen atom, a halogen atom, an alkyl group containing from 1 through 6 carbon atoms, and an alkenyl group containing from 1 through 6 carbon atoms, each o independently represents an integer of from 0 through 4, p represents an integer of from 1 through 11, and R⁴ represents any one of a saturated or unsaturated, straight-chained or branched hydrocarbon group that contains from 1 through 12 carbon atoms and may contain an ether bond, a substituted phenylene group represented by general formula (1-1) below, and a divalent group represented by general formula (1-2) below,

<General Formula (1-1)>

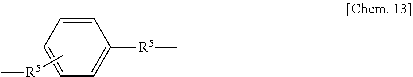

where in general formula (1-1), R⁵ represents any one of a methylene group and an ethylene group,

<General Formula (1-2)>

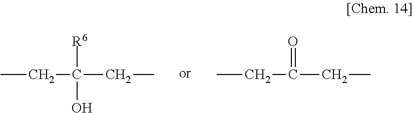

where in general formula (1-2), R⁶ represents any one of a hydrogen atom and an alkyl group containing from 1 through 4 carbon atoms.

<2> The thermosensitive recording medium according to <1>,

wherein the content of the diphenylsulfone compound represented by general formula (1) is 0.1 parts by mass or greater but 2.5 parts by mass or less relative to 1.0 part by mass of the 4-hydroxy-4′-benzyloxydiphenylsulfone.

<3> The thermosensitive recording medium according to <1> or <2>,

wherein the content of the diphenylsulfone compound represented by general formula (1) is 0.3 parts by mass or greater but 1.5 parts by mass or less relative to 1.0 part by mass of the 4-hydroxy-4′-benzyloxydiphenylsulfone.

<4> The thermosensitive recording medium according to any one of <1> to <3>,

wherein the diphenylsulfone compound represented by general formula (1) is a compound represented by structural formula A below,

<Structural Formula A>

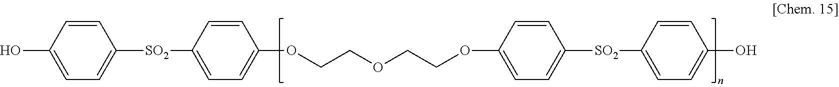

where in structural formula A, n is a numerical value of from 1 to 11.

<5> The thermosensitive recording medium according to any one of <1> to <4>, including

a protective layer provided on the thermosensitive recording layer and containing a modified polyvinyl alcohol.

<6> The thermosensitive recording medium according to <5>,

wherein the modified polyvinyl alcohol is at least one selected from the group consisting of a diacetone-modified polyvinyl alcohol, an acetoacetyl-modified polyvinyl alcohol, and a carboxylic acid-modified polyvinyl alcohol.

<7> The thermosensitive recording medium according to any one of <1> to <6>, including

an undercoat layer provided between the support and the thermosensitive recording layer and containing hollow particles.

<8> The thermosensitive recording medium according to <7>,

wherein a ratio of hollowness of the hollow particles is 50% or greater.

<9> The thermosensitive recording medium according to <7> or <8>,

wherein a content of the hollow particles in the undercoat layer is 20% by mass or greater but 50% by mass or less.

<10> The thermosensitive recording medium according to any one of <1> to <9>, further including

a viscous layer provided on a surface of the support opposite to the surface provided with the thermosensitive recording layer.

<11> The thermosensitive recording medium according to any one of <1> to <9>, further including:

a release layer as a topmost layer on the surface of the support provided with the thermosensitive recording layer; and

a viscous layer provided on a surface of the support opposite to the surface provided with the thermosensitive recording layer.

<12> An article including

the thermosensitive recording medium according to any one of <1> to <11>.

The thermosensitive recording medium according to any one of <1> to <11> and the article according to <12> can solve the various problems in the related art and achieve the object of the present invention.

1. A thermosensitive recording medium, comprising:
a support; and
a thermosensitive recording layer that is provided over at least one surface of the support and comprises a leuco dye, a first developer, and a second developer,
wherein the first developer and the second developer are 4-hydroxy-4′-benzyloxydiphenylsulfone and a diphenylsulfone compound of formula (1), respectively, and
wherein a content of the diphenylsulfone compound is less than 3.0 parts by mass relative to 1.0 part by mass of the 4-hydroxy-4′-benzyloxydiphenylsulfone, 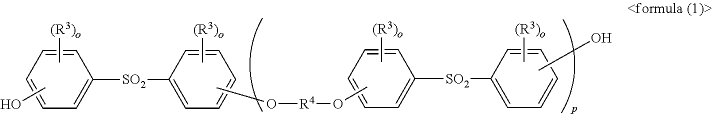
where R³ represents any one of a hydrogen atom, a halogen atom, an alkyl group that comprises from 1 through 6 carbon atoms, and an alkenyl group that comprises from 1 through 6 carbon atoms, each o independently represents an integer of from 0 through 4, p represents an integer of from 1 through 11, and R⁴ represents any one of a saturated or unsaturated, straight-chained or branched hydrocarbon group that comprises from 1 through 12 carbon atoms and optionally comprises an ether bond, a substituted phenylene group of formula (1-1), and a divalent group of formula (1-2), 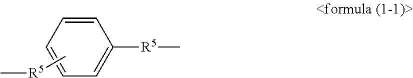
where R⁵ represents any one of a methylene group and an ethylene group, 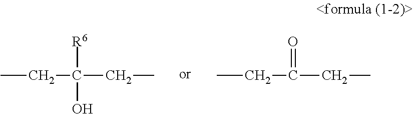
where R⁶ represents any one of a hydrogen atom and an alkyl group that comprises from 1 through 4 carbon atoms.

2. The thermosensitive recording medium according to claim 1,
wherein the content of the diphenylsulfone compound is 0.1 parts by mass or greater but 2.5 parts by mass or less relative to 1.0 part by mass of the 4-hydroxy-4′-benzyloxydiphenylsulfone.

3. The thermosensitive recording medium according to claim 1,
wherein the content of the diphenylsulfone compound is 0.3 parts by mass or greater but 1.5 parts by mass or less relative to 1.0 part by mass of the 4-hydroxy-4′-benzyloxydiphenylsulfone.

4. The thermosensitive recording medium according to claim 1,
wherein the diphenylsulfone compound comprises a compound of formula A: 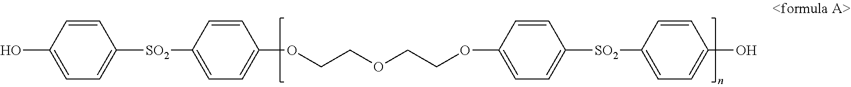
wherein n is a numerical value of from 1 to 11

5. The thermosensitive recording medium according to claim 1, further comprising
a protective layer that is provided on the thermosensitive recording layer and comprises a modified polyvinyl alcohol.

6. The thermosensitive recording medium according to claim 5,
wherein the modified polyvinyl alcohol comprises at least one selected from the group consisting of a diacetone-modified polyvinyl alcohol, an acetoacetyl-modified polyvinyl alcohol, and a carboxylic acid-modified polyvinyl alcohol.

7. The thermosensitive recording medium according to claim 1, further comprising
an undercoat layer that is provided between the support and the thermosensitive recording layer and comprises hollow particles.

8. The Thermosensitive recording medium according to claim 7,
wherein a ratio of hollowness of the hollow particles is 50% or greater.

9. The thermosensitive recording medium according to claim 7,
wherein a content of the hollow particles in the undercoat layer is 20% by mass or greater but 50% by mass or less.

10. The thermosensitive recording medium according to claim 1, further comprising
a viscous layer provided on a surface of the support opposite to the surface provided with the thermosensitive recording layer.

11. The thermosensitive recording medium claim 1, further comprising:
a release layer as a topmost layer on the surface of the support provided with the thermosensitive recording layer; and
a viscous layer provided on a surface of the support opposite to the surface provided with the thermosensitive recording layer.

12. An article, comprising:
a thermosensitive recording medium,
wherein the thermosensitive recording medium comprises:
a support and
a thermosensitive recording layer that is provided over at least one surface of the support and comprises a leuco dye, a first developer, and a second developer,
wherein the first developer and the second developer are 4-hydroxy-4′-benzyloxydiphenylsulfone and a diphenylsulfone compound of formula (1), respectively, and
wherein a content of the diphenylsulfone compound is less than 3.0 parts by mass relative to 1.0 part by mass of the 4-hydroxy-4′-benzyloxydiphenylsulfone, 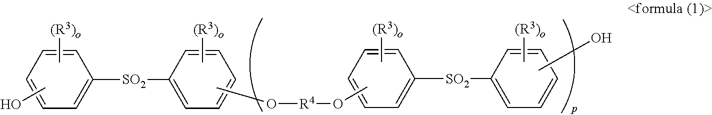
where R³ represents any one of a hydrogen atom, a halogen atom, an alkyl group that comprises from 1 through 6 carbon atoms, and an alkenyl group that comprises from 1 through 6 carbon atoms, each o independently represents an integer of from 0 through 4, p represents an integer of from 1 through 11, and R⁴ represents any one of a saturated or unsaturated, straight-chained or branched hydrocarbon group that comprises from 1 through 12 carbon atoms and optionally comprises an ether bond, a substituted phenylene group represented by the following the following general formula (1-1), and a divalent group of formula (1-2), 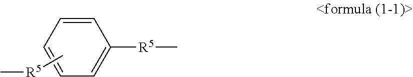
where R⁵ represents any one of a methylene group and an ethylene group, 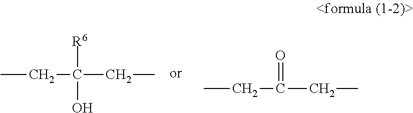
where R⁶ represents any one of a hydrogen atom and an alkyl group that comprises from 1 through 4 carbon atoms.
